(19) United States
(12) Patent Application Publication
(10) Pub. No.: US 2021/0375407 A1
(43) Pub. Date: Dec. 2, 2021

(54) DIAGNOSTIC GENOMIC PREDICTIONS BASED ON ELECTRONIC HEALTH RECORD DATA

(71) Applicant: The Trustees of Columbia University in the City of New York, New York, NY (US)

(72) Inventors: Kai Wang, Princeton, NJ (US); Chunhua Weng, New York, NY (US)

(21) Appl. No.: 16/648,336

(22) PCT Filed: Oct. 2, 2018
(86) PCT No.: PCT/US2018/053855

§ 371 Date: Mar. 18, 2020

Related U.S. Application Data
(60) Provisional application No. 62/568,851, filed on Oct. 6, 2017.

(51) Int. Cl. G16H 10/60; G16B 20/00; G16B 50/10; G16H 50/20; G06F 40/10; G06F 40/30; G06F 40/40

(52) U.S. Cl. CPC G16H 10/60; G16B 20/00; G16B 50/10; G06F 40/40; G06F 40/10; G06F 40/30; G16H 50/20

(57) ABSTRACT

Disclosed are methods, devices, systems, circuits, media, and other implementations that include a method including accessing electronic health record data for a patient, performing natural language processing on the electronic health record data to extract biomedical concepts, processing the biomedical concepts to obtain phenotype terms, normalizing the phenotype terms to generate normalized phenotype terms, and identifying based on the normalized phenotype terms one or more candidate genes responsible for one or more medical conditions causing at least some of the biomedical concepts extracted from the electronic health record data.

### CROSS-REFERENCE TO RELATED APPLICATIONS

This application claims the benefit of U.S. Provisional Application No. 62/568,851, entitled “AUTOMATED TOOL FOR DIAGNOSTIC GENOMIC PREDICTIONS BASED ON ELECTRONIC HEALTH RECORD DATA” and filed Oct. 6, 2017, the content of which is incorporated herein by reference in its entirety.

### STATEMENT REGARDING FEDERALLY SPONSORED RESEARCH

This invention was made with government support under HG006465 and HG008680 awarded by the National Institutes of Health (NIH). The government has certain rights in the invention.

### BACKGROUND

Traditionally, the diagnostic workup of individuals with suspected monogenic disease has relied on sequential testing using a battery of genetic and biochemical studies, incurring substantial time and financial costs while the causal etiology remains elusive. In addition, the diagnostic uncertainty, ambiguity regarding appropriate clinical management, and repeated medical evaluations during this “diagnostic odyssey” pose a weighty emotional and psychosocial burden on both affected individuals and their families.

Since they were first reported to resolve a case with an undiagnosed genetic disease, next-generation sequencing (NGS) methods, including whole-exome sequencing (WES) and whole-genome sequencing (WGS), have been quickly established as a scalable method for efficiently generating a molecular diagnosis. The diagnostic yield of WES ranges from 25% to 51% and has been shown to be cost effective when used as a first-line test. However, the challenge of interpreting the vast amount of sequence data generated by genome-wide testing still hinders the broad clinical utilization of this technology.

### SUMMARY

Disclosed are systems, methods, circuits, devices, media and other implementation for a novel integrative framework that uses nuanced EHR narratives for deep phenotyping of patients represented by standardized phenotype terms, performs gene-ranking using such phenotype terms and facilitate genetic diagnosis in diagnostic labs when exome or genome data is available. This framework uses a modular architecture so that, in some embodiments, the natural language processing and gene-ranking components can be replaced with alternative methods.

Electronic health records (EHRs) can serve as a rich, integrated source of phenotype information. Automatic extraction and recognition of phenotypes from EHR narratives can accelerate the adoption and utilization of phenotype-driven efforts to improve genomic diagnostics and gene discovery. Such automation is especially needed in the context of diagnostic sequencing, given that most clinical information is submitted as a copy of the free-text clinical evaluation note or as a short, relatively nonspecific clinical description (such as “congenital heart disease”). Moreover, the current proprietary nature of NGS informatics pipelines implemented in various laboratories impedes standardized processes for variant interpretation. This deficiency can be partially addressed via direct, systematic integration of phenotypes extracted from EHRs, therefore improving information synthesis at the time of interpretation.

The EHR-Phenolyzer implementations described herein provide an automated EHR-narrative-based phenotyping pipeline, to allow phenotype-based gene prioritization. The present disclosure includes a discussion of tests and evaluation of some EHR-narrative-based phenotyping pipelines that demonstrate the efficacy of EHR-derived deep phenotyping information in facilitating genetic diagnosis from, for example, WES data. The testing and evaluations performed also provide a comparative analysis of natural language processing (NLP) systems in parsing EHR narratives for phenotype extraction and normalization to evaluate the ability of EHR-Phenolyzer to analyze real-world EHR data and prioritize candidate genes from WES of positively diagnosed individuals.

Thus, in some variations, a method is provided that includes accessing electronic health record data for a patient, performing natural language processing on the electronic health record data to extract biomedical concepts, processing the biomedical concepts to obtain phenotype terms, normalizing the phenotype terms to generate normalized phenotype terms, and identifying based on the normalized phenotype terms one or more candidate genes responsible for one or more medical conditions causing the biomedical concepts extracted from the electronic health record data.

Embodiments of the method may include at least some of the features described in the present disclosure, including one or more of the following features.

Processing the biomedical concepts may include recognizing the biomedical concepts for disease phenotypes using semantic knowledge resources, including one or more of, for example, UMLS (Unified Medical Language System), and/or HPO (Human Phenotype Ontology).

Identifying the one or more candidate genes may include prioritizing the one or more candidate genes responsible for one the or more medical conditions causing the biomedical concepts extracted from the electronic health record data.

Prioritizing the one or more candidate genes may include ranking the one or more identified candidate genes. Ranking the one or more identified candidate genes may include ranking the one or more candidate genes based on degree of matching between the normalized phenotype terms and respective descriptors associated with the one or more candidate genes.

Accessing the electronic health record data may include determining data quality of the electronic health record data, selecting portions of the electronic health record data based, at least in part, on the determined data quality, and representing the selected portions of the electronic health record data in a pre-determined format for further analysis.

Processing the biomedical concepts may include applying text processing to the electronic health record data at the document level and the sentence level, with the text processing comprising performing one or more of, for example, semantic knowledge-based and/or machine-learning based concept recognition to obtain the phenotype terms.

Performing the one or more of the semantic knowledge-based or machine-learning based concept recognition to obtain the phenotype terms may include performing one or more of, for example, i) analyzing negation status associated with recognized phenotype terms, ii) analyzing phenotype existence for the patient or a family member of the patient to rule-out non-patient phenotype, iii) identifying modifiers associated with the recognized phenotype terms, iv) analyzing temporal properties associated with the recognized phenotype terms, and/or v) analyzing temporal relationships among one or more phenotype terms for the patient.

Normalizing the phenotype may include performing semantic knowledge-based concept normalization.

Normalizing the phenotype terms may include normalizing the phenotype terms using human phenotypes ontology (HPO) definitions to generate the normalized phenotype terms.

The method may further include obtaining clinical exome or genome data representative of one or more genetic profiles of the patient, and determining at least one gene from the one or more identified candidate genes responsible for the one or more medical conditions based on the clinical exome or genome data and on the normalized phenotype terms provided to a gene-ranking tool.

Performing the natural language processing on the electronic health record data to extract biomedical concepts may include performing the natural language processing (NLP) through multiple independent NLP platforms to produce respective multiple lists of extracted biomedical concepts. Identifying the one or more candidate genes may include providing the respective multiple lists of extracted biomedical concepts to a gene-ranker to generate multiple lists of candidate genes.

The method may further include ranking each of the generated multiple lists of candidate genes, and deriving a composite ranked list of candidate genes based on the ranked multiple lists of candidate genes.

Performing natural language processing on the electronic health record data may include performing natural language processing on clinical patient notes from the electronic health record data.

In some variations, a medical analysis system is provided that includes a communication module to access electronic health record data for a patient stored in a data storage device, and a natural language processing engine. The natural language processing engine is configured to perform natural language processing on the accessed electronic health record data to extract biomedical concepts, process the biomedical concepts to obtain phenotype terms, and normalize the phenotype terms to generate normalized phenotype terms. The medical analysis system further includes a genetic analyzer configured to identify based on the normalized phenotype terms one or more candidate genes responsible for one or more medical conditions causing the biomedical concepts extracted from the electronic health record data.

Embodiments of the medical analysis system may include at least some of the features described in the present disclosure, including at least some of the features described above in relation to the method, as well as one or more of the following features.

The natural language processing engine configured to process the biomedical concepts may be configured to recognize the biomedical concepts for disease phenotypes using semantic knowledge resources, including one or more of, for example, UMLS (Unified Medical Language System), and/or HPO (Human Phenotype Ontology).

The genetic analyzer may include a gene-ranking tool to prioritize the one or more candidate genes responsible for one the or more medical conditions causing the biomedical concepts extracted from the electronic health record data.

The gene-ranking tool configured to prioritize the one or more candidate genes may be configured to rank the one or more identified candidate genes based on degree of matching between the normalized phenotype terms and respective descriptors associated with the one or more candidate genes.

The genetic analyzer may further be configured to obtain clinical exome or genome data representative of one or more genetic profiles of the patient, and determine at least one gene from the one or more identified candidate genes responsible for the one or more medical conditions based on the clinical exome or genome data and on the normalized phenotype terms provided to the gene-ranking tool.

The system may further include at least one other communication module to access the electronic health record data for the patient, and at least one other natural language processing engine configured to generate at least one other independent set of normalized phenotype terms provided to the genetic analyzer. The genetic analyzer may be configured to identify the one or more candidate genes based further on the at least one other independent set of normalized phenotype terms.

In some variations, an apparatus is provided that includes means for accessing electronic health record data for a patient, means for performing natural language processing on the electronic health record data to extract biomedical concepts, means for processing the biomedical concepts to obtain phenotype terms, means for normalizing the phenotype terms to generate normalized phenotype terms, and means for identifying based on the normalized phenotype terms one or more candidate genes responsible for one or more medical conditions causing the biomedical concepts extracted from the electronic health record data.

In some variations, non-transitory computer readable media is provided, that includes computer instructions, executable on one or more processor-based devices, to access electronic health record data for a patient, perform natural language processing on the electronic health record data to extract biomedical concepts, process the biomedical concepts to obtain phenotype terms, normalize the phenotype terms to generate normalized phenotype terms, and identify based on the normalized phenotype terms one or more candidate genes responsible for one or more medical conditions causing the biomedical concepts extracted from the electronic health record data.

Embodiments of the apparatus and the computer readable media include at least some of the features described in the present disclosure, including at least some of the features described above in relation to the method and to the medical analysis system.

Other features and advantages of the invention are apparent from the following description, and from the claims.

### BRIEF DESCRIPTION OF THE DRAWINGS

These and other aspects will now be described in detail with reference to the following drawings.

FIG. 1 is a schematic diagram of an example medical analysis system configured to identify and/or prioritize candidate genes based on phenotype data determined from electronic medical record data.

FIG. 2 is a flow diagram of an example process for identifying a gene with a causal variant based on clinical exome or genome data, and based phenotype data derived from a patient's electronic health record data.

FIG. 3 is a flowchart of an example procedure to identify possible genes responsible for medical conditions or ailments afflicting an individual.

FIG. 4 is a schematic diagram of an example computing system.

FIGS. 5A-B are example illustrations showing resultant output for two NLP systems extracting phenotype terms from natural language descriptions in an example clinical note.

FIG. 6 includes a graph of gene ranking performances for various system implementations used in a first study performed at a first site.

FIG. 7 includes a graph of gene ranking performances for various system implementations used in a second study performed at a second site.

Like reference symbols in the various drawings indicate like elements.

### DESCRIPTION

The disclosure presented herein is directed to a high-throughput EHR phenotype extraction and analysis pipeline. A natural language processing system is used to extract biomedical concepts from clinical records and generate relevant phenotype terms. These terms are then normalized using, for example, Human Phenotypes Ontology, and can then be fed into an analysis module (also referred to herein as a genetic analyzed and/or the “EHR-Phenolyzer” implementation) configured to associate potential causative genes to patient phenotypes (e.g., symptoms, signs, comorbidities, etc.), to thus identify candidate genes. This tool can therefore aid in informed selection of genetic tests to order and improve the efficiency of genetic diagnosis. This technology was successfully used to identify causative genes in several case studies and a larger scale pilot study is ongoing. EHR-Phenolyzer can allow comprehensive utilization of the wealth of data available within EHRs and facilitate the implementation of genomic medicine. Some of the implementations described herein (e.g., the EHR-Phenolyzer) thus provide a high-throughput EHR framework for extracting and analyzing phenotypes. The EHR-Phenolyzer implementations extract and normalize Human Phenotype Ontology (HPO) concepts from EHR narratives, and then prioritizes genes with causal variants on the basis of the HPO-coded phenotype manifestations. In one study to evaluate the efficacy of the implementations described herein, the EHR-Phenolyzer was applied to records of 28 pediatric individuals with confirmed diagnoses of monogenic diseases. The genes with causal variants were ranked among the top 100 genes selected by EHR-Phenolyzer for 16/28 individuals (p<2.2×10¹⁶), supporting the value of phenotype-driven gene prioritization in diagnostic sequence interpretation. To further assess the generalizability of the approaches developed and studied herein, the implementations were applied to an independent EHR dataset of ten individuals with a positive diagnosis from a different institution. Through several retrospective case studies, combined analyses of genotype data and deep phenotype data from EHRs were shown to expedite genetic diagnoses. The EHR-Phenolyzer implementations described herein can thus leverage EHR narratives to automate phenotype-driven analysis of clinical exomes or genomes, to the broader implementation of genomic medicine.

Thus, in some embodiments, a medical analysis system is provided that includes a data storage device (e.g., implementing a local or remote database/data repository system) to store electronic health record data for one or more patients, and a natural language processing system (implemented using a processor-based device such as a server, or a local device) configured to access electronic health record data for a patient, perform natural language processing on the electronic health record data to extract biomedical concepts (e.g., from clinical patient notes of the electronic health record data), process the biomedical concepts to select phenotype terms, and normalizing the phenotype terms (e.g., using human phenotypes ontology (HPO) definitions) to generate standard phenotype representations. The medical analysis system further includes a genetic analyzer configured to identify, based on the normalized phenotype terms, at least some of one or more candidate genes responsible for one or more medical conditions causing the biomedical concepts extracted from the electronic health record data. In some examples, the genetic analyzer may include a gene-ranking tool to prioritize the one or more candidate genes responsible for one the or more medical conditions causing the biomedical concepts extracted from the electronic health record data. Such a ranking tool may be configured to rank the one or more identified candidate genes based on degree of matching between the normalized phenotype terms and respective descriptors associated with the one or more candidate genes. In some variations, the genetic analyzer may further be configured to obtain clinical exome or genome data representative of one or more genetic profiles of the patient, and determine at least one gene from the one or more identified candidate genes responsible for the one or more medical conditions based on the clinical exome or genome data and on the normalized phenotype terms provided to the gene-ranking tool.

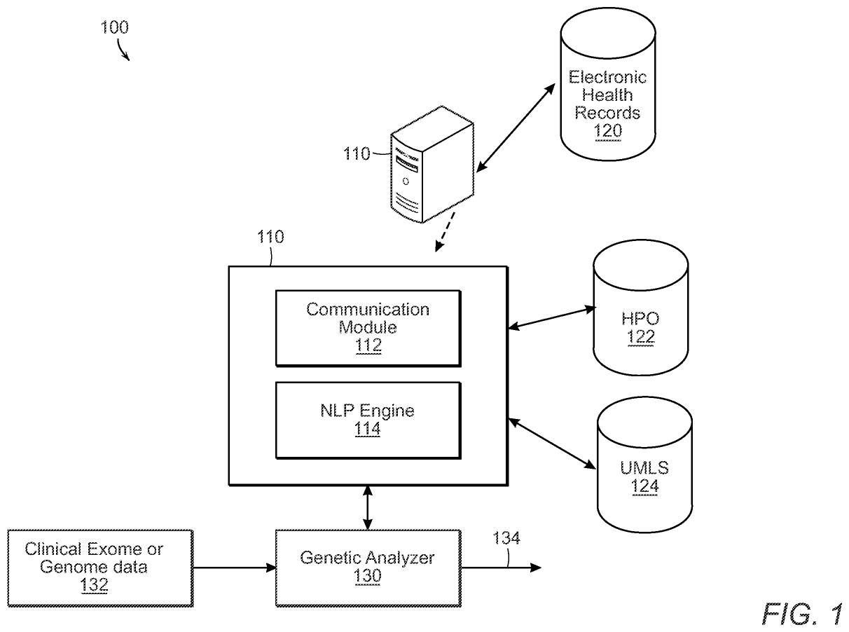

More particularly, with reference to FIG. 1, a schematic diagram is provided of an example medical analysis system 100 configured to identify and/or prioritize candidate genes that are potentially responsible for one or more medical conditions a patient may have, based (at least in part) on phenotype data determined/derived medical records for the patient. As illustrated, the system 100 includes a controller 110 (which in the example embodiments of FIG. 1 is implemented using a computing device) in communication with a repository/database 120 that stores, inter alia, electronic health record data for one or more patients. Such health record data may include lab results, doctor notes, clinical assessments, and any other type of medical data record that may be determined and maintained for a particular patient. The database 120 may be locally accessible by the controller 110 (and, in fact, may be housed within the same housing containing the various circuitries and modules constituting the controller 110), or may be a remote repository/database accessible directly (through wired or wireless connections) or via a network (e.g., a private network, or a public network such as the internet). The controller 110 may thus include, in some embodiments, a communication interface 112 (e.g., comprising one or more transceivers and/or modems) to establish communication channels with the remote repository/database 120, and/or to establish communication channels with other devices (including with other data repositories, as will become apparent below). Such a communication interface may be configured (under the control of a controller) to access the repository/database 120. Accessing the electronic health record data may include sending a request for particular data records, or passively receiving a communication initiated by the repository/database 120 (or initiated by some other device or party). Requests handled by the repository/database 120 result in retrieval of the requested electronic records from storage of the repository/database 120, and communicating them to the controller 110.

In some embodiments, accessing of the electronic health data record may include some pre-processing of the data being accessed, performed either at the repository 120 (e.g., by a local controller at the repository 120) or by the controller 110. Such pre-processing operations may include determining data quality of the electronic health record data, selecting portions of the electronic health record data based, at least in part, on the determined data quality, and representing the selected portions of the electronic health record data in a pre-determined format for further analysis. For example, the data quality determination criteria may include screening for data that is more recent (e.g., earlier than some pre-determined date), screening for narrative data only (such as doctor's counseling notes) while excluding various types of information (e.g., lab works) that cannot provide meaningful phenotype information. In another example, the electronic health record is analyzed to identify appropriate data types (e.g., determining portions of the record(s) that include lab results, clinical notes, etc.) and selecting for further processing and analysis only those portions matching pre-determined data types (e.g., using for further analysis only narrative content provided in clinical counseling notes). Having identified data portions from a particular electronic health record that are to be excluded (or, alternatively, kept), a revised record may be generated and formatted according to the format required by the controller 110 and/or other downstream devices/modules. For example, the newly generated record (or revised record) may be formatted as a vector-based patient representation to facilitate subsequent downstream computational analysis.

As further depicted in FIG. 1, the controller 110 additionally includes a natural language processing engine 114, which may be implemented as a software-based application, a hardware-based application, or a combination thereof. The natural language processing engine 114 may be implemented using commercial NLP applications (such as a MedLee™ or MetaMap™) or customized NLP applications. The implemented NLP application is generally configured to perform natural language processing on the accessed electronic health record data to extract biomedical concepts from clinical patient notes, process the biomedical concepts to obtain phenotype terms, and normalize the phenotype terms (e.g., using human phenotypes ontology, or HPO, or some other pre-defined set of definitions) to generate normalized phenotype terms. Generally, natural language processing is applied to data sources to process human language for meaning (semantics) and structure (syntax). NLP can further differentiate meaning of words/phrases and larger text units based on the surrounding semantic context (pragmatics). Syntactical processors assign or “parse” units of text to grammatical categories or “part-of-speech” (noun, verb, preposition, etc.) Semantic processors assign units of text to lexicon classes to standardize the representation of meaning. Text communications are said to be “tokenized” when discrete units of text are classified according to their semantic and syntactical categories. NLP systems may also use classifiers, also referred to as ontologies, which are sets of concepts that are used to parse, or otherwise analyze input data sources. Two examples of ontologies that may be used in conjunction with the system 100 to process biomedical concepts include UMLS (Unified Medical Language System), and/or HPO (Human Phenotype Ontology). HPO is configured to allow computable representations of phenotype concepts with terms sourced from clinically oriented medical literature and gene-disease databases, such as Online Mendelian Inheritance in Man (OMIM). Additional efforts such as the Monarch Initiative and PhenomeCentral use high-quality crowd-sourced phenotype information to further enrich and refine computational abilities embedded in HPO. HPO may thus serve as a standardized terminology and a phenotype-genotype knowledge database. The various ontologies used by the system 100 may have been downloaded to the local machine performing the NLP (i.e., the controller 110), or, alternatively, such ontologies may be stored remotely (e.g., at data repositories 122 and 124) and accessed, as needed, by the controller 110 (e.g., via the communication module 112) during performance of the natural language processing on the electronic health record data obtained by the controller 110.

The NLP engine 114 may thus be configured, in some implementations, to apply NLP processing to the electronic health record data at the document level and the sentence level (or at lower or higher granular levels), with the NLP processing including performing one or more of, for example, semantic knowledge-based or machine-learning based concept recognition to obtain the phenotype terms. In embodiments in which the NLP engine 114 includes a machine learning system, such a system is configured to iteratively analyzes training input data and the input data's corresponding output, and derive functions or models that cause subsequent inputs to produce outputs consistent with the machine's learned behavior. For example, initially a training data set provided to the machine learning system of the NLP engine 114 may be used to define the response of the learning machine. The training data set can be as extensive and comprehensive as desired, or as practical. At the end of the learning process, the learning machine is ready to accept input corresponding to one or more subject matter concepts that expand on existing ontologies that are available to the controller 110. In some embodiments, a machine learning implementation of the NLP engine 114 may be configured to process input data based on pre-defined procedures (e.g., adaptive processing and/or computations).

In some examples, the learning machine may be implemented as a neural network. Neural networks are in general composed of multiple layers of transformations (multiplications by a “weight” matrix), each followed by a linear or nonlinear function. The linear transformations are learned during training by making small changes to the weight matrices that progressively make the transformations more helpful to the final classification task (e.g., classification of electronic health record data into one or more biomedical concepts such as phenotypes). The layered network may include convolutional processes which are followed by pooling processes along with intermediate connections between the layers to enhance the sharing of information between the layers. Examples of neural networks include convolutional neural network (CNN), recurrent neural networks (RNN), etc. Convolutional layers allow a network to efficiently learn features that are invariant to an exact location in a data set by applying the same learned transformation to subsections of the entire data set. Other examples of learning engines that may be implemented as part of the NLP engine 114 may include a support vector machine, decision trees techniques, regression techniques, and/or other types of machine learning techniques. Such machine learning techniques and/or implementations may be used, for example, to determine (or to facilitate the determination) of the “closeness” of matches between the input data sources and the ontology attributes (and/or their associated processing rules) against which the input data sources are compared.

As part of the NLP processing, some of the following operations may be performed.
Analyzing negation status associated with recognized phenotype terms. For example, the NLP engine 114 may be configured to recognize whether a biomedical concept (e.g., a symptom of condition) is indicated to be present or not present with respect to the associated patient,
Analyzing phenotype existence for the patient or a family member of the patient to rule-out non-patient phenotypes,
Identifying modifiers associated with recognized phenotype terms, e.g., severity, certainty/likelihood, frequency, etc.,
Analyzing temporal properties associated with the recognized phenotype terms (in order to determine when and for how long a patient has had, or has exhibited, the particular recognized phenotype), and/or
Analyzing temporal relationships among one or more phenotype terms for the patient.

The NLP engine 114 is generally configured to normalize phenotype terms recognized or identified via its initial NLP processing so that downstream components of the system 100 receive a standardized set of terms that those components (including, for example, a genetic analyzer 130 configured to identify candidate causative genes that may be responsible for various medical conditions or symptoms indicated in the electronic health record data) can more easily and efficiently process and analyze the data provided to them by the NLP engine 114. Accordingly, in such embodiments, the NLP processor may be configured to performing semantic knowledge-based concept normalization. The normalization may be based on matching recognized concepts (which were identified via NLP processing performed using biomedical ontologies such as UMLS or HPO) to a more limited/narrower set of concepts (that may have been customized for the downstream analyzer 130) based on semantic similarity/closeness of the biomedical concepts to the normalized set of phenotypes, based on a pre-determined set of rules, based on machine-learning processes (e.g., implemented using a learning machine such as the one(s) that may be used by the NLP engine 114, and in which the learning machine is trained to produce normalized output responsive to recognized/identified phenotypes produced by the upstream NLP processing performed on the electronic health record data), etc.

As noted, and as further depicted in FIG. 1, coupled to the output of the NLP engine 114 is the genetic analyzer 130. The genetic analyzer 130 receives the normalized phenotype output produced by, for example, the NLP engine 114 of the controller 110, and identifies, based on the normalized phenotype terms (e.g., normalized according to an HPO ontology), one or more candidate genes responsible for one or more medical conditions causing the biomedical concepts extracted from the electronic health record data. The analyzer 130 may be integrally connected to the controller 110 (i.e., it may be housed in the same unit as the NLP engine 114, or run using the same processor that executes the NLP engine 114), or it may be a separate unit that can communicate (via communication interfaces) with the controller 110. The genetic analyzer 130 (also referred to as the “Phenolyzer”) takes a discrete list of phenotype terms (e.g., normalized by the NLP engine 114, in some embodiments) as an input and generates a list of candidate genes weighted by the chance of being associated with the phenotype. The analyzer 130 generally does not require any genotype data for ranking of candidate genes. In some embodiments, the NLP engine 114 may be configured to first interpret submitted disease phenotypes/terms into a set of HPO/OMIM descriptors. The tool may use CTD Medic vocabulary, Disease Ontology, precompiled OMIM disease synonyms, Human Phenotype Ontology database, and/or OMIM descriptors. The Human Phenotype Ontology (HPO) is a standardized vocabulary bank of phenotypic abnormalities to describe presentations of human pathologies. The terminology for HPO is developed based on Online Mendelian Inheritance in Man (OMIM) database.

Upon interpretation of submitted phenotypes/terms, the tool queries each disease name in the pre-compiled gene-disease databases. The Phenolyzer may incorporate, in some embodiments, a list of gene-disease databases, pre-compiled from several data sources, including OMIM, Orphanet, ClinVar, Gene Reviews and GWAS catalog. Each time a gene is found to be directly associated with a disease, a score is calculated. As a result, the tool finds all the genes (“seed genes”) that have a reported association with known diseases. The seed genes are then expanded to include related genes. Several types of gene-gene relationship logic are used, such as exhibiting a protein-protein interaction, sharing a biological pathway or gene family, or transcription regulation or being regulated by another gene. The seed gene set is grown based on four different types of gene relationship databases, namely, HPRD, NCBI's Biosystem, HGNC Gene Family and HTRI databases. At a final stage of the analysis, a final gene set with normalized scores is generated. The results can be visualized as a gene-gene-disease interaction network, a bar plot that lists top 500 genes and their scores, disease tag cloud, etc. In an example case study, SCN8A gene was successfully identified as the most relevant gene based on patient's phenotype data from physician's report that was converted into short phrases/HPO terms, and submitted to the Phenolyzer, with this finding being confirmed by WES data. In a second example case study, comprehensive analysis of an extended pedigree, including genomics filtering on WGS data and phenotypic prioritization of candidate genes using Phenolyzer, was performed. In this particular example study, the pedigree involved probands with Prader-Willi Syndrome (PWS), Hereditary Hemochromatosis (HH), dysautonomia-like symptoms, Tourette Syndrome (TS) and other illnesses and included 14 individuals from 3 generations. Nine members of the family underwent WGS. The Phenomizer tool was used to rank the highest priority diagnosis based on the clinical features of one of the probands. The implemented Phenolyzer tool accurately revealed the diagnosis of PWS for that proband and how genes in the deletion regions identified by WGS are linked towards the phenotypes represented by HPO terms. The Phenolyzer revealed the relationship between a potentially causal variant and HH in another proband by combining data from the subject's genomic and phenotypic profiles.

Accordingly, in some embodiments, the analyzer configured to identify the one or more candidate genes is configured to prioritizing the one or more candidate genes responsible for one the or more medical conditions causing the biomedical concepts extracted from the electronic health record data. Prioritizing the one or more candidate genes may include ranking the one or more identified candidate genes. Ranking the one or more identified candidate genes may include ranking the one or more candidate genes based on degree of matching between the normalized phenotype terms and respective descriptors associated with the one or more candidate genes.

In some examples, the analyzer 130 may also be configured to determine one or more causative genes, from the candidate genes that are identified based on the phenotype terms identified through NLP operations performed by the controller 110, by further using exome or genome data that is obtained by the analyzer 130. For example, as shown in FIG. 1, the analyzer may optionally receive exome or genome data 132 (e.g., from a remote server, from a DNA sequencer analyzing DNA samples of the particular patient for whom the electronic health record data was analyzed by the controller 110, etc.), and cross-reference the two sources of data (the genes identified via NLP operations, and gene sequences included in the exome or genome data) to identify a likely (or possibly multiple likely) genes that are potentially responsible for medical conditions affecting the patient. Thus, in such embodiments, the genetic analyzer 130 is further configured to obtain clinical exome or genome data representative of one or more genetic profiles of the patient, and determine at least one gene from the one or more identified candidate genes responsible for the one or more medical conditions based on the clinical exome or genome data and on the normalized phenotype terms provided to the gene-ranking tool. In some embodiments, the analyzer 130 may be a DNA sequencer that can receive DNA specimen for a patient and generate locally exome or genome DNA profiles, and then use normalized phenotype terms received from the controller (i.e., the output of the NLP engine 114 of the controller 110) to determine at least one gene (as shown in block 134 of FIG. 1) that potentially is responsible for a medical condition affecting the patient.

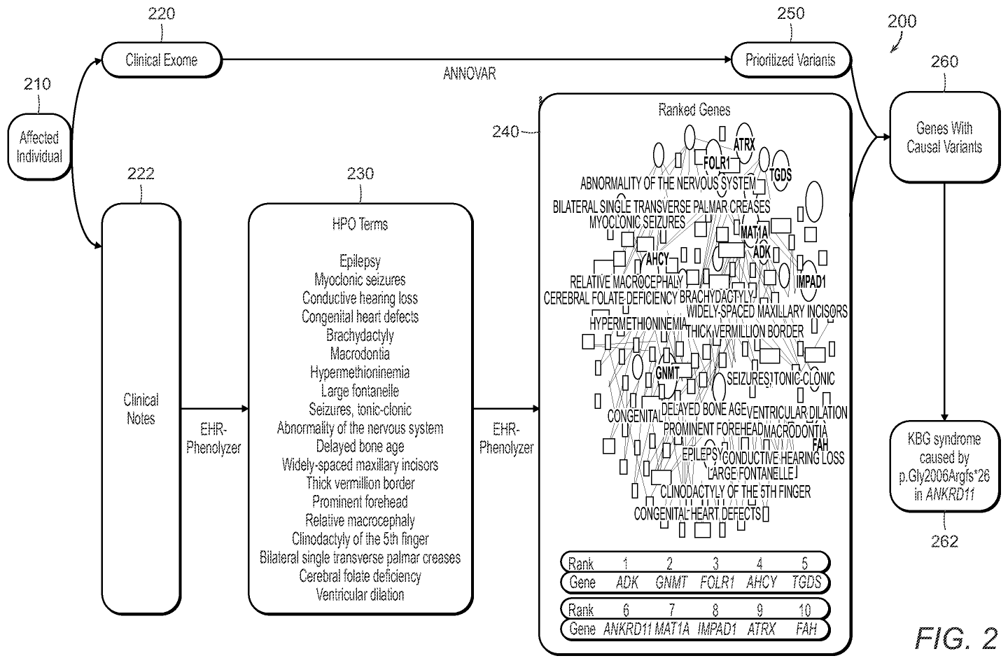

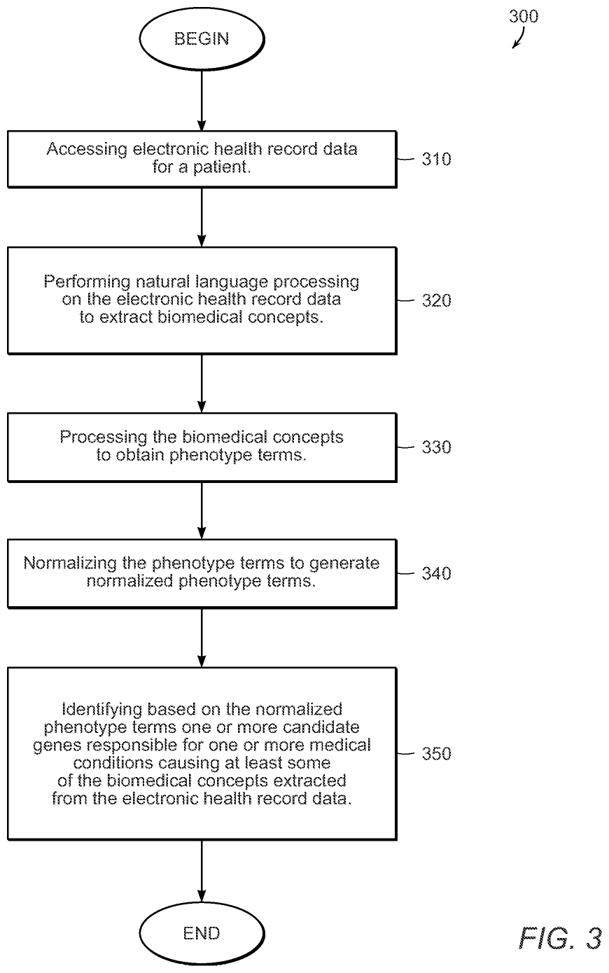

FIG. 2 is a flow diagram of an example process 200 for identifying a gene with a causal variant based on clinical exome or genome data 220 for an affected individual 210, and based on an analysis of the electronic health record data 222 (in this case the clinical notes) for the affected individual. As shown, the clinical notes are processed (by an NLP tool such as the EHR-phenolyzer tool implemented herein) to produce phenotype terms 230 (e.g., HPO terms), that are then provided to the analyzer tool (such as the analyzer 130 of the system 100) to produce identified and ranked genes 240 associated with the various phenotype terms considered by the analyzer. The clinical exome or genome data (which may be generated at the analyzer 130 if the analyzer also includes a DNA sequencer device) can be analyzed by a tool (e.g., such as ANNOVAR) to identify genetic variants 250 (in reference to an exome or genome database) that may be responsible for conditions affecting the individual, and prioritize those variants. Based on the ranked genes 240 produced using clinical notes, and the prioritizes genetic variants 250 produced through analysis of exome or genome data, genes with causal variants (e.g., that potentially are responsible for a medical condition(s) affecting the individual 210) are identified and ranked/prioritized 260 (e.g., based on which ranked gene in the ranked genes list 240 is associated with the largest number of biomedical concepts gleaned or identified from the clinical notes). Some possible genes that may have been identified in the list 240 and that cannot be matched to the prioritized variants 250 may be eliminated from further consideration. In the example embodiment of FIG. 2, the process 200 resulted, based on a combined genotype and phenotype analysis, in a molecular diagnosis, at 262, of KBG Syndrome in an Individual with a Frameshift Mutation in ANKRD11.

In a study of the efficacy of using a process such as that depicted in FIG. 2, it was demonstrated that genes with causal variants can be ranked much higher than other genes when phenotype information extracted from EHRs is used. To further demonstrate the applicability of this method in real-world settings to facilitate the identification of disease-causing variants, several published cases were analyzed for which a combined analysis was performed of genotype data (VCF files) and clinical descriptions from the methods sections of the published manuscripts. It was observed that the clinical descriptions in scientific manuscripts were professionally edited and could be of higher quality than typical EHR narratives, but extracting HPO terms from the public case reports posed challenges similar to those faced in EHR settings. The first case study (analyzed by a process similar to that illustrated in FIG. 2) was of an individual diagnosed with KBG syndrome, which is a rare autosomal-dominant genetic condition characterized by intellectual disability, seizures, and distinct facial, hand, and skeletal features. A de novo, single-nucleotide insertion in ankyrin repeat domain 11 (ANKRD11 [MIM: 611192]) was previously identified as the disease-causing variant through the analysis of trio WES data. In the current study, parental information was not used to infer de novo variants, but instead only the exome data of the proband was analyzed. All missense, nonsense, stop-gain, frameshift, and splice variants with an allele frequency<1×10⁻⁵ were identified in gnomAD, which is a publicly available allele-frequency database of 123,136 exomes. There were 459 prioritized variants in this list (typically <100 variants are identified after filtering, but these exome data were generated on the Ion Torrent platform, resulting in a large number of potential false-positive calls). Using phenotype terms derived from the EHR-Phenolyzer pipeline depicted in FIG. 1 (e.g., through the controller 110 and the analyzer 130) that used a MetaMap-based engine, an analysis based only on the electronic health record data ranked ANKRD11 as #6. After the overlap between the Phenolyzer list and the prioritized variant list was compared, ANKRD11 was ranked first, providing a molecular diagnosis of KBG syndrome even without parental information. This result was replicated by using the EHR-Phenolyzer pipeline with MedLEE as the NLP engine, yielding identical results.

A second case study was focused on a sibling pair (brother and sister) both affected by progressive cognitive decline starting from 6 years of age. A compound-heterozygous mutations in N-acetylalpha-glucosaminidase (NAGLU [MIM: 609701]) was previously identified, leading to a genetic diagnosis of Sanfilippo syndrome (mucopolysaccharidosis IIIB). Biochemical tests confirmed the complete loss of activity of alpha-N-acetylglucosaminidase (encoded by NAGLU) in both individuals. In the current study, shared variants between the siblings were not analyzed or filtered for, and instead each individual's exome was analyzed separately. An allele-frequency threshold of 0.01 was used to account for the possibility that causal variants for recessive conditions could be observed in public databases with a relatively high allele frequency. For the sister, using phenotype terms derived from a EHR-Phenolyzer pipeline with a MetaMap engine, the approach described herein ranked NAGLU as #42 among all human genes. After comparing the overlap between this list and the prioritized list of 885 variants, NAGLU was ranked as #1 for the observed phenotypes. For the brother, NAGLU was ranked as #201, and the intersection between this list and the prioritized list of 892 variants increased the rank to #1. Therefore, in both cases, the gene with causal variant was successfully identified, and yielded a molecular diagnosis through combined analysis of genotypes and phenotypes. Similar results were obtained with the EHR-Phenolyzer pipeline with MedLEE as the NLP engine, confirming that the combination of EHR-Phenolyzer and exome or genome data can often significantly expedite and improve molecular diagnosis of monogenic disorders.

Turning back to FIG. 1, it is to be noted that in some embodiments, the system 100 may include multiple pipelines implemented through a combination of the controller 110 and an analyzer 130. Each such pipeline, which may perform the phenotype-based NLP analysis independently using different NLP engines and/or different ontologies, yields a respective list of prioritize genes. In some embodiments, while each pipeline may use a different NLP engine implementation, a common genetic analyzer may be used to receive the separately produced normalized phenotype terms, based on which separate lists of ranked genes may be generated. Subsequently, the analyzer 130 (or some other downstream device or module) may process/analyze the multiple sets of normalized phenotype terms (produced independently by each of the multiple pipelines) and produce a composite list of ranked genes (using an averaging or weighing operations applied to the respective lists). The composite list may be compared to an exome or genome data to winnow the list to a smaller set of ranked genes (possibly identifying the likely genes implicated as causing the medical condition from which the affected individual suffers). Thus, in such embodiments, the system, such as the system 100 depicted in FIG. 1, may further include at least one additional controller comprising another communication module to access the electronic health record data for the patient, and another natural language processor configured to generate another independent set of normalized phenotype terms provided to the genetic analyzer. The genetic analyzer can then be configured to identify the one or more candidate genes based further on the other independent set of normalized phenotype terms.

With reference next to FIG. 3, a flowchart of an example procedure 300 to identify possible genes responsible for medical conditions or ailments affecting an individual is shown. The procedure 300 includes accessing 310 electronic health record data for a patient. As noted, accessing may include establishing a communication channel with a remote repository or database (such as the database 120 of FIG. 1), and requesting retrieval, and transmission of, relevant electronic health record for the patient back to a controller on which a pipeline to analyze the data, in the manner described herein, is provided. In some situations, the data repository storing the electronic health record data may already be locally stored at the machine on which the pipeline is implemented. In some embodiments, accessing the electronic health record data may include determining data quality (e.g., data type, completeness of record, and various other criteria of quality) of the electronic health record data, selecting portions of the electronic health record data based, at least in part, on the determined data quality, and representing the selected portions of the electronic health record data in a pre-determined format (e.g., as a revised record with pre-defined field, as a data vector, etc.) for further analysis. Such pre-processing may be performed at the electronic health record data repository, or at the controller implementing the pipeline (e.g., after retrieval of the EHR data from a remote repository).

As further illustrated in FIG. 3, the procedure 300 also includes performing 320 natural language processing on the electronic health record data to extract biomedical concepts. In some examples, the natural language processing may be performed on a portion of the electronic helat3h record data, e.g., only on the clinical patient notes. In some examples, the procedure may use multiple pipelines to perform the NLP operations. In such examples, performing the natural language processing on the electronic health record data may include performing the natural language processing (NLP) through multiple independent NLP platforms to produce respective multiple lists of extracted biomedical concepts, and providing the respective outputs of such NLP operations to an analyzer that can independently identify, based on each of the generated output resulting from the independent NLP pipelines, prioritized candidate genes. In such embodiments, the procedure 300 may further include ranking each of the generated multiple lists of candidate genes, and deriving a composite ranked list of candidate genes based on the ranked multiple lists of candidate genes.

The procedure 300 additionally includes processing 330 the biomedical concepts to obtain phenotype terms. Processing the biomedical concepts may include recognizing the biomedical concepts for disease phenotypes using semantic knowledge resources, including one or more of, for example, UMLS (Unified Medical Language System), and/or HPO (Human Phenotype Ontology). In some embodiments, processing the biomedical concepts may include applying text processing to the electronic health record data at the document level and the sentence level, with the text processing comprises performing one or more of semantic knowledge-based or machine-learning based concept recognition to obtain the phenotype terms. Examples of the one or more semantic knowledge-based or machine-learning based concept recognition may include performing one or more of, for example: 1) analyzing negation status associated with recognized phenotype terms, 2) analyzing phenotype existence for the patient or a family member of the patient to rule-out non-patient phenotype, 3) identifying modifiers associated with recognized phenotype terms, 4) analyzing temporal properties associated with the recognized phenotype terms, and/or analyzing temporal relationships among one or more phenotype terms for the patient.

The procedure 300 further includes normalizing 340 the phenotype terms to generate normalized phenotype terms. For example, normalizing the phenotype terms may include normalizing the phenotype terms using human phenotypes ontology (HPO) definitions to generate the normalized phenotype terms. Normalizing the phenotype may include performing semantic knowledge-based concept normalization.

The procedure 300 also includes identifying 350 based on the normalized phenotype terms one or more candidate genes responsible for one or more medical conditions causing at least some of the biomedical concepts extracted from the electronic health record data. In some examples, identifying the one or more candidate genes may include prioritizing the one or more candidate genes responsible for one the or more medical conditions causing the at least some of the biomedical concepts extracted from the electronic health record data. In some embodiments, prioritizing the one or more candidate genes may include ranking the one or more identified candidate genes. Such ranking may include ranking the one or more candidate genes based on degree of matching between the normalized phenotype terms and respective descriptors associated with the one or more candidate genes.

In some implementations, the procedure may further include using exome or genome data to facilitate a more accurate and reliable identification of the candidate genes that may be causing the medical condition or ailment of the patient. Thus, in such implementations, the procedure may additionally include obtaining clinical exome or genome data representative of one or more genetic profiles of the patient, and determining at least one gene from the one or more identified candidate genes responsible for the one or more medical conditions based on the clinical exome or genome data and on the normalized phenotype terms provided to a gene-ranking tool.

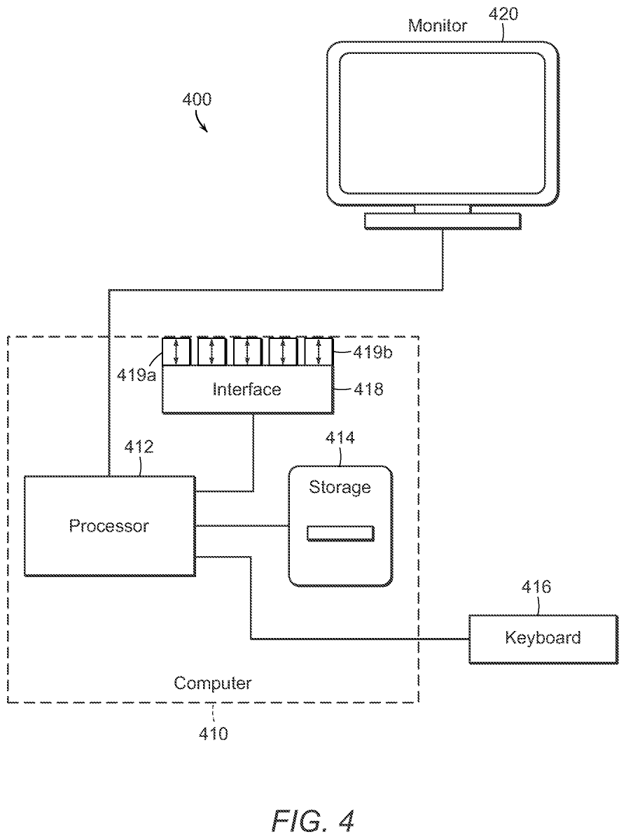

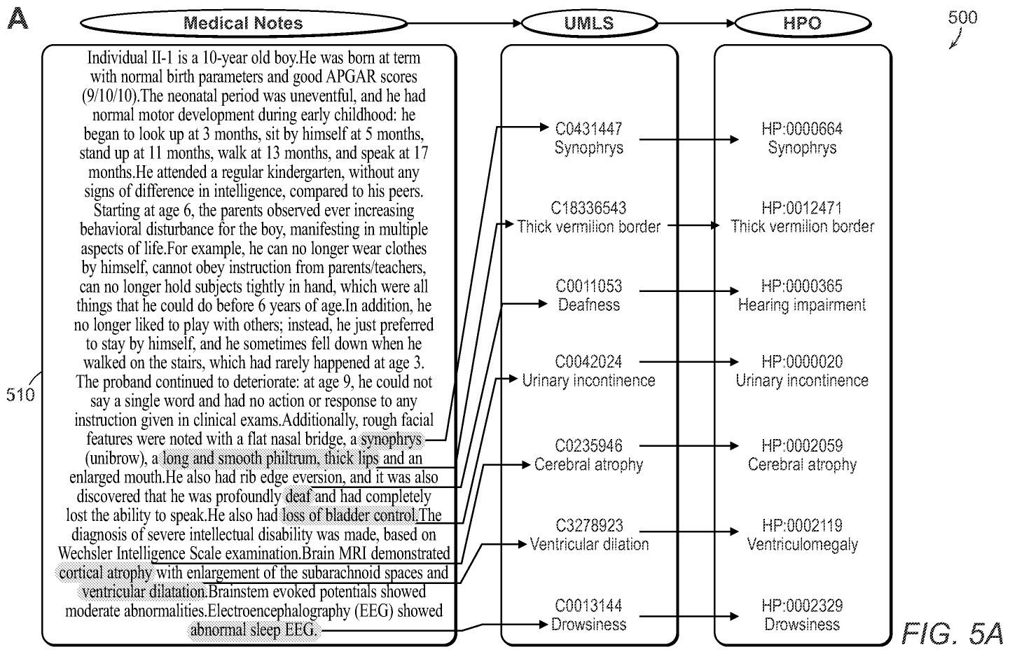

Performing the procedures described herein may be facilitated by a processor-based computing system. With reference to FIG. 4, a schematic diagram of an example computing system 400 is shown. Part or all of the computing system 400 may be housed in, for example, a computing device (e.g., server, a user station, a handheld mobile device, etc.), or may comprise part or all of the servers, nodes, access points, storage device, and all other devices or systems described herein, including any of the devices and modules depicted in FIG. 1. The computing system 400 includes a computing-based device 410 such as a personal computer, a specialized computing device, a controller, and so forth, that typically includes a central processor unit 412. In addition to the CPU 412, the system includes main memory, cache memory and bus interface circuits (not shown). The computing-based device 410 may include a mass storage device 414, such as a hard drive and/or a flash drive associated with the computer system, to store data (e.g., electronic health record data, ontologies and dictionaries used for NLP, etc.) and instructions executable on a processor or controller such as the CPU 412. The computing system 400 may optionally further include a keyboard, or keypad, 416, and/or a monitor 420, e.g., an LCD (liquid crystal display) monitor, that may be placed where a user can access them. In some embodiments, the keyboard and monitor may be located remotely from the computing-based device 410 (e.g., when the computing-based device is a central server that operators can access remotely).

The computing-based device 410 is configured to facilitate, for example, the implementation of one or more of the procedures/processes/techniques described herein, including to access electronic health record data and ontologies, perform natural language processing to determine or recognize phenotype terms, and identify candidate genes based at least in part on the phenotype terms (or a normalized version thereof). The mass storage device 414 may thus include a computer program product that when executed on the computing-based device 410 causes the computing-based device to perform operations to facilitate the implementation of the procedures described herein. The computing-based device may further include peripheral devices to provide input/output functionality. Such peripheral devices may include, for example, a CD-ROM drive and/or flash drive, or a network connection, for downloading related content to the connected system. Such peripheral devices may also be used for downloading software containing computer instructions to enable general operation of the respective system/device. For example, as illustrated in FIG. 4, the computing-based device 410 may include an interface 418 with one or more interfacing circuits, such interfacing circuits may include, for example, a wireless port that includes transceiver circuitry, a network port (such as a port 419a) with circuitry to interface with one or more network device (including with network devices that include a radio-access network (RAN), a WiFi network, and so on), a powerline interface (such as an interface 419b) to interface with devices via powerlines, and other types of interfaces to provide/implement communication with remote devices. Alternatively and/or additionally, in some embodiments, special purpose logic circuitry, e.g., an FPGA (field programmable gate array), a DSP processor, or an ASIC (application-specific integrated circuit) may be used in the implementation of the computing system 400. Other modules that may be included with the computing-based device 410 are speakers, a sound card, a pointing device, e.g., a mouse or a trackball, by which the user can provide input to the computing system 400. The computing-based device 410 may include an operating system.

Computer programs (also known as programs, software, software applications or code) include machine instructions for a programmable processor, and may be implemented in a high-level procedural and/or object-oriented programming language, and/or in assembly/machine language. As used herein, the term “machine-readable medium” refers to any non-transitory computer program product, apparatus and/or device (e.g., magnetic discs, optical disks, memory, Programmable Logic Devices (PLDs)) used to provide machine instructions and/or data to a programmable processor, including a non-transitory machine-readable medium that receives machine instructions as a machine-readable signal.

Memory may be implemented within the computing-based device 410 or external to the device. As used herein the term “memory” refers to any type of long term, short term, volatile, nonvolatile, cache or non-cache, or other memory device type, and is not to be limited to any particular type of memory or number of memories, or type of media upon which memory is stored.

If implemented in firmware and/or software, the functions may be stored as one or more instructions or code on a computer-readable medium. Examples include computer-readable media encoded with a data structure and computer-readable media encoded with a computer program. Computer-readable media includes physical computer storage media. A storage medium may be any available medium that can be accessed by a computer. By way of example, and not limitation, such computer-readable media can comprise RAM, ROM, EEPROM, CD-ROM or other optical disk storage, magnetic disk storage, semiconductor storage, or other storage devices, or any other medium that can be used to store desired program code in the form of instructions or data structures and that can be accessed by a computer; disk and disc, as used herein, includes compact disc (CD), laser disc, optical disc, digital versatile disc (DVD), floppy disk and Blu-ray disc where disks usually reproduce data magnetically, while discs reproduce data optically with lasers. Combinations of the above are also be included within the scope of computer-readable media.

The following are a few illustrative examples of implementations, and operations performed therewith, that were used to test and evaluate the NLP engine and genetic analyzer pipeline configurations and approaches developed to facilitate diagnosis of genetic disorders. In one example, a pipeline was developed using the NLP tools MedLEE and MetaMap, to extract phenotype concepts from genetics counseling notes. As part of the pre-processing performed on electronic health record data, the most recent clinical genetic consultation notes were selected, before the WES-confirmed genetic diagnoses, under the assumption that they were more complete and accurate than older consultation notes. In a primary cohort (28 individuals), four had genetic evaluation notes, which included information regarding diagnostic genetic findings, because a prior diagnostic workup and/or sequencing from another institution or laboratory had become available by the time of their evaluation visit. For these individuals, the evaluation note included the documentation of genetic test results and a short description of the genetic diagnosis. To prevent such text from biasing the phenotyping process, these portions may be removed before applying the NLP parsing. Additional types of pre-processing operations included removing the “review of systems” section (if present) from the evaluation notes because many of these sections contained un-parsable, template-based structured tables that became corrupted or lost during the extraction of EHR data to, for example, plain text. In addition, these sections typically contained tandem repeats of negated concepts (i.e., “no lymphadenopathy” or “no murmurs”), which add little value to the recognition of phenotype concepts. Because phenotype (e.g., HPO) concepts aim to represent mostly pertinent positive findings and only prominently salient negative findings (i.e., “absent speech”), the removal of this section can be justified. For MedLEE, such pre-processing was not necessary because the build-in section-detection methods can be used to systematical delineate the sections via XML parsing.

The implementations that were tested and evaluated also included various NLP system configuration choices. For MetaMap, a local installation of MetaMap was selected by using the latest supported version of the Unified Medical Language System (UMLS; 2016AA release). Starting from the UMLS 2015AB release, the entire HPO database had been integrated into UMLS, which permits making the configuration to restrict output to HPO concepts (command-line parameter “-R ‘HPO’”). In addition, a review of the expert-selected phenotypes revealed that the HPO phenotype concepts frequently belonged to a limited number of UMLS semantic types. In order to prevent an excessive number of non-relevant terms from being mapped, seven (7) UMLS semantic types were chosen that effectively represented the larger class of expert-curated HPO concepts. These included “congenital abnormality” (T019), “genetic function” (T045), “laboratory procedure” (T059), “laboratory or test result” (T034), “pathologic function” (T046), “disease or syndrome” (T047), and “finding” (T033). Specifically, the options “-I -p -J -K -8 -conj cgab, genf, lbpr, lbtr, patf, dsyn, fndg -R ‘HPO’” were used in the application of MetaMap.

For MedLEE, the NLP engine's lexicon was loaded with HPO terms and synonyms available via UMLS (version 2017AA). Text files were processed, outputting an XML file with tagged tokens regarding information in the clinical note section, token information, HPO concept(s) identified, and certainty and negation information. A Python script using an XML-parsing library (lxml) was used to extract all HPO concepts. The concepts found in the “review of systems” section were excluded without pre-processing.

Configurations of all NLP tools were set to allow for multiple suggestions for a given text phrase as semantic concept recognition was being performed. The scripts for recognition of phenotype concepts and output parsing for each NLP tool are accessible at the EHR-Phenolyzer GitHub repository. The output of each tool was a list of HPO concepts (via HPO concept IDs and/or preferred terms) for each given clinical note input as plain text. To handle multiple instances for each concept within a given note, only unique HPO concepts were selected.

The performance of gene prioritization was evaluated by using Phenolyzer and Phenomizer, which can both accept HPO terms as input and generate a ranked gene list as output. For Phenolyzer, command-line tools available at the Phenolyzer GitHub repository (version v.0.2.0) was used. The “-f -p -ph -logistic -addon DB_DISGENET_GENE_DISEASE_SCORE, DB_GAD _GENE_DISEASE_SCORE-addon weight 0.25” argument was used in the command-line tool to ensure consistency with the web server implementation of the Phenolyzer.

For analysis with Phenomizer, the web server available at the Phenomizer website was used because a command-line tool is not publicly available. For each individual, HPO terms were manually entered into the web interface for analysis. The “any” mode of inheritance was selected for the diagnosis, and if the number of input HPO terms was larger than five, the “symmetric” mode was added into the analysis. After Phenomizer generated results in the web interface, the raw text output file was manually downloaded for further processing by a custom Python script to get the gene rankings.

A fourth independent cohort containing 20 individuals with CKD was analyzed to evaluate whether EHR phenotypes can help classify disease subtypes. First, EHR-Phenolyzer was applied on the medical notes to generate HPO terms, and a hierarchical clustering method was then used to study the categorization of individuals with CKD. In the clustering analysis, the “complete linkage” was used as the agglomeration method and “Euclidean distance” to calculate the distance between any two individuals. Only individuals with diagnostic genes ranked within the top 50 and with phenotype terms found in at least two individuals but not all were used in the clustering analysis.

Two methods of selecting EHR data for phenotyping were tested: (1) comprehensive chart review (reviewing the EHRs of each person and synthesizing phenotype concepts from various clinical notes, laboratory tests, imaging results, and pathology reports), and (2) targeted review of genetic notes (retrieving the most recent medical genetic consultation note before WES and synthesizing the phenotypes from the note). The latter method examines a much smaller subset of phenotype concepts than the first approach but has the clear advantage of being more efficient and more likely to be fully automatable on EHRs. To evaluate whether targeted review of genetic notes alone is sufficient in practice, the performances of gene prioritization by these two approaches on 28 affected individuals (for whom diagnostic mutations were identified by WES) were compared. For each approach, a list of phenotype terms was generated and provided to the Phenolyzer tool to generate a ranked gene list that allowed examination of where the gene with causal variants ranked. It was determined that the ranking performances were effectively identical between the two methodologies (paired t test p=0.44 for testing differences in performance); more than 50% of confirmed genetic diagnosis occurred within the top 100 predicted candidate genes by Phenolyzer. Therefore, it can be concluded that the latest genetic notes can reliably be used before diagnostic exome sequencing as the data source for gene ranking.

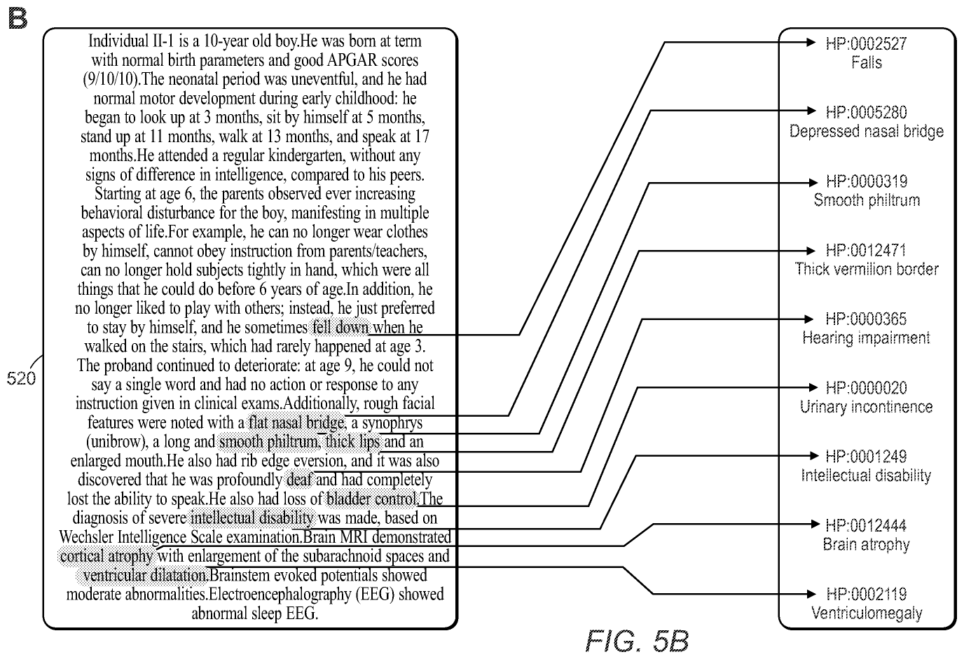

The performance of NLP Tools in extracting phenotype terms was also evaluated in the course of the testing and studies performed for some of the implementations described herein. The types of EHR narratives that contain the documentation of phenotypes for genetic disorders were first identified. The text of the identified narratives were then provided to NLP systems to extract phenotype concepts and normalize them by using the HPO. The Phenolyzer implementation then analyzed these HPO terms to identify related genes with causal variants. Two different NLP systems were adapted, namely, MedLEE and MetaMap, to process genetic notes from EHRs and extract and normalize phenotype concepts by using HPO. FIGS. 5A and 5B are illustrations 500 showing how the two NLP systems extract phenotype terms from natural language descriptions in clinical notes. Particularly, FIGS. 5A and 5B include an initial (prior to normalization) NLP output 510 performed by MetaMap on a clinical note, and another output 520 performed by MedLEE on the same clinical note. In general, both NLP systems tended to generate more terms (on average, 17.6 and 19.4 terms for MetaMap and MedLEE, respectively) than manual extraction by human experts (11.0 terms).

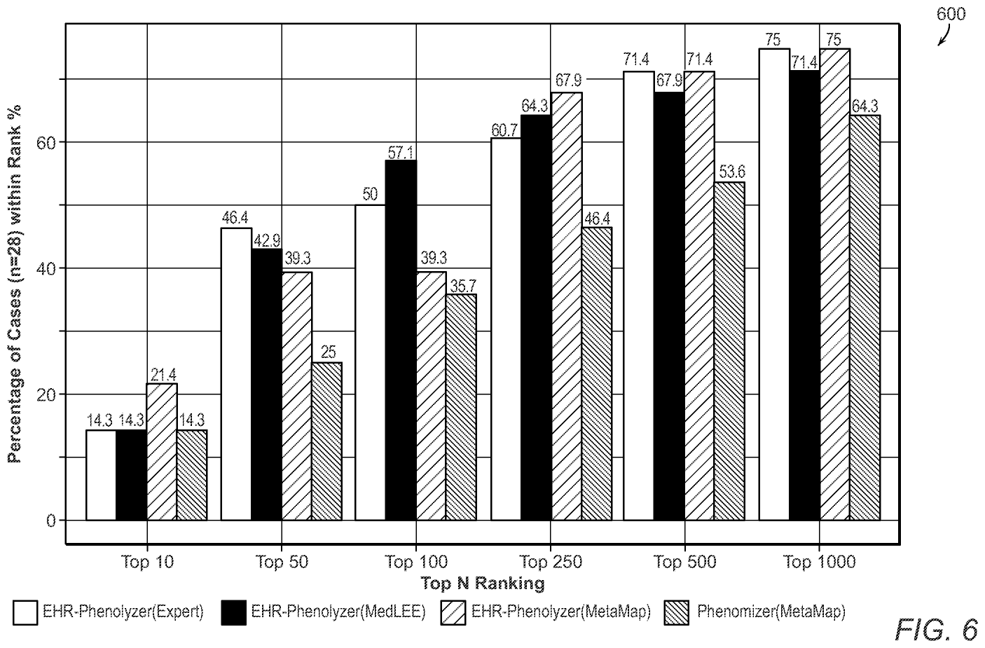

Next, the Phenolyzer implementation's ability to rank genes with causal variants by using phenotype terms (compiled by experts or extracted by the NLP methods MetaMap and MedLEE) was assessed. The ranking performances of these methods, when used in a first study at a first site (Columbia University) are shown in a graph 600 of FIG. 6. The NLP systems performed similarly to experts, although each NLP system generated more terms than the experts did. The results showed that 39.3%-57.1% of gene candidates could be ranked within the top 100 genes and that 71.4%-75.0% of gene candidates could be ranked within the top 1,000 genes, both on the basis of only the phenotype concepts derived from the EHR. To evaluate different phenotype-based gene-prioritization tools, gene ranking results were also included from Phenomizer on MetaMap-generated HPO terms. An analysis of the results demonstrated that Phenolyzer performs favorably against Phenomizer on the same set of HPO terms, most likely because Phenolyzer's gene-prioritization procedures incorporate multiple levels of prior biological knowledge (this may be, in part, because the Phenomizer system is designed for disease diagnosis rather than gene prioritization, so it might not have performed optimally in the testing and evaluation conducted). The fact that about 50% of diagnoses can be narrowed to the top 100 genes on the basis of only phenotype information documented in the EHR is remarkable, especially because this performance can be achieved by completely automated phenotype-concept-recognition methods (i.e., MetaMap or MedLEE). It is believed that deep phenotypes from EHR data are valuable with the increasing adoption of genomics testing. Improving the prior probability of a diagnosis increases the positive predictive value of a test, although current genomic testing methods tend to forgo this step. Therefore, systematic integration of EHR-phenotype-based gene prioritization before variant interpretation can potentially improve workflow efficiency and help reach clinically valid results while improving diagnostic yield.

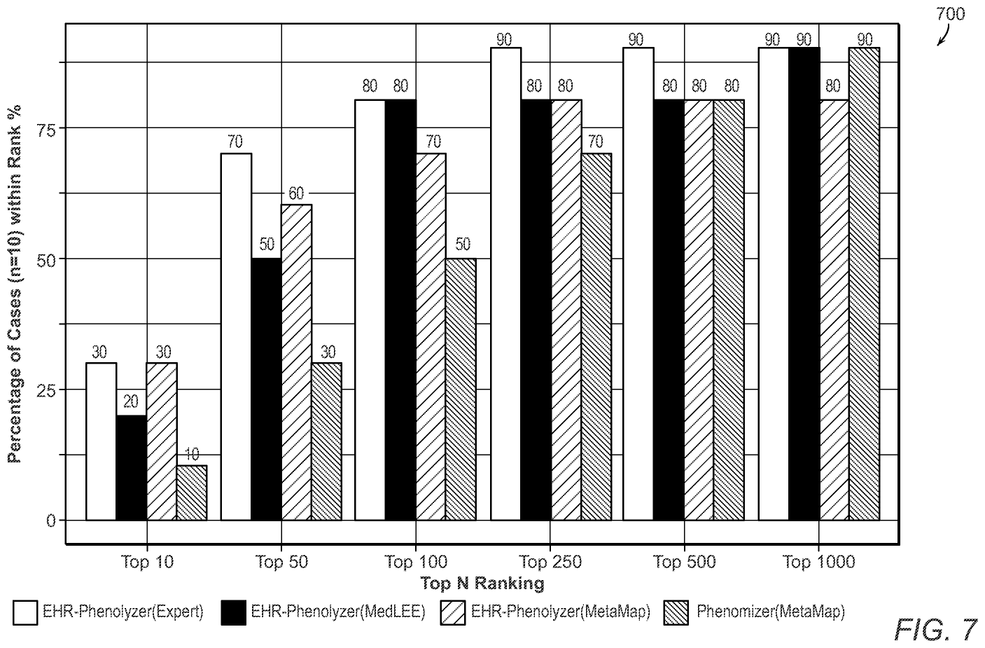

Another part of the testing and evaluation involved external validation of automated phenotype description and gene prioritization. The same pipelines were applied by using clinical notes written by genetic counselors from the Mayo Clinic. Information on ten affected individuals, together with confirmed genetic diagnoses in the genes cystic fibrosis transmembrane conductance regulator (CFTR [MIM: 602421]), peripheral myelin protein 22 (PMP22[MIM: 601097]), DM1 protein kinase (DMPK [MIM: 605377]), dynamin 1(DNM1 [MIM: 602377]), coagulation factor VIII (F8 [MIM: 300841]), fibrillin 1 (FBN1 [MIM: 134797]), KAT8 regulatory NSL complex subunit 1 (KANSL1 [MIM: 612452]), NPC intracellular cholesterol transporter 1 (NPC1 [MIM: 607623]), sodium voltage-gated channel alpha subunit 1 (SCN1A [MIM: 182389]), and SOS Ras/Rac guanine nucleotide exchange factor 1 (SOS1 [MIM:182530]), was provided. The ranking results are shown in FIG. 7. The results are comparable to the ranking performance obtained for the original study (performed at Columbia University). Thus, the analysis on the secondary-site validation data confirmed that the EHR-Phenolyzer approach can be used in different institutions with diverse sets of informatics infrastructure as long as an automated procedure for extracting clinical notes can be implemented in each site.

Next, to examine how clinical phenotypes are currently used in real-world settings to facilitate genetic diagnosis of people with rare monogenic diseases, EHR data on 46 affected individuals was examined, all of whom were assessed by a medical geneticist or genetic counselor at Columbia University affiliated hospitals in an outpatient setting. This set of clinical notes, together with the corresponding molecular pathology reports, should be highly informative on the real-world use of clinical phenotype information in the context of genetic testing. It was determined that 15 of 46 affected individuals did not undergo diagnostic genetic testing, the reasons for which were lack of known reimbursable tests (n=7), lack of insurance (n=2), refusal by family members (n=2), lack of testing records in EHRs (n=1), and other undescribed reasons (n=3). Among the 31 affected individuals who underwent genetic testing, the genetic tests used were clinical microarray (n=11), PCR (n=2), single-gene Sanger sequencing (n=5), targeted panel (n=2), clinical exome (n=9), and undescribed (n=2). Diagnostic results were detected in 11 of the 31 (35.5%) affected individuals; 7 (63.6%) of these individuals had been diagnosed via clinical WES.

To understand how phenotype information is used in current clinical practice to assist in genetic diagnosis, the genetic diagnostic reports for each of the 31 affected individuals were manually examined. These diagnostic reports were generally provided as scanned PDF files from the following clinical labs: Ambry Genetics (n=4), GeneDx (n=12), Columbia University Personalized Genomic Medicine Laboratory Hospital lab (n=3), Integrated Genetics (n=5), LabCorp (n=4), Mayo Clinic (n=1), and unspecified (n=2). It was determined that 19 (61%) of the 31 diagnostic reports contained no indication of a clinical phenotypes, suggesting that clinical phenotypes were either not provided to diagnostic labs or not used by diagnostic labs in making a diagnosis. Among the 12 genetic diagnostic reports with information about the indication for testing, the indication was most commonly listed in an unstructured sentence or paragraph format ( 8/12 [67%]); in the others, it was listed simply as ICD codes ( 3/12 [25%]) or as the single general term “diagnostic” ( 1/12 [8%]). The indication was compared with clinical phenotypes inferred by MetaMap or MedLEE from clinical notes in EHRs. With the exception of one individual for whom there were no detailed notes by the genetic counselor, the clinical phenotypes from EHRs were consistently more comprehensive and detailed than those provided in the indication, which could improve the diagnostic yield for clinical labs. For the 11 individuals with positive results from genetic diagnostic testing, the study next examined whether deep phenotypes from EHRs can facilitate prioritization of candidate genes, similarly to what had been done on the primary and secondary cohorts described above. It was found that the genes with causal variants were ranked among the top 100 or top 1,000 genes for over 50% or 91%, respectively, of the affected individuals, again suggesting that EHR-derived phenotype information could greatly increase the efficiency of genetic diagnosis. Furthermore, similar to previous observations, it was also determined that Phenolyzer outperformed Phenomizer on this set of affected individuals, justifying the use of computational tools specifically designed for phenotype driven gene prioritization.

Another aspect that was investigated was whether EHR-Phenolyzer can be useful for discerning specific genetic forms of a broader category of disease with CKD as a model. Discerning hereditary versus acquired etiologies of CKD oftentimes has a substantial impact on clinical prognosis and management; however, the two can be indistinguishable by traditional diagnostics alone. Because many hereditary nephropathies display substantial genetic and phenotypic heterogeneity, gene panels or genome-wide testing can help diagnose individuals with a suspected monogenic renal disease. The EHRs of a set of 20 individuals with CKD was evaluated and confirmed genetic diagnosis. It was determined that EHR-Phenolyzer (based on either MedLEE or MetaMap) worked especially well for this set of individuals in that it ranked the genes with causal variants within the top ten for nearly half of them. This observation can be attributed to two reasons: (1) given that these individuals were recruited from a large academic referral center for renal disease, many were already well characterized and had been diagnosed by traditional methods (e.g., kidney biopsy for Alport syndrome), so genetic testing served as a merely confirmatory test; and (2) the specificity of the kidney-related phenotypes listed in these individuals' EHRs would also restrict the number of candidate genes. A hierarchical clustering was additionally performed on this set of individuals on the basis of the presence or absence of specific phenotype terms. For the 13 individuals with diagnostic genes ranked within the top 50 by EHR-Phenolyzer, it was found that the individuals with the same genes with causal variants, such as the two individuals with uromodulin (UMOD [MIM: 191845]) mutations and the four individuals with collagen type IV alpha 5 chain (COL4A5 [MIM: 303630]) mutations, tended to be clustered together according to the phenotype terms. Nevertheless, there were also scenarios in which affected individuals with the same diagnostic genes had quite distinct phenotypes from each other (such as the individuals with COL4A4 [MIM: 120131] mutations), which suggests that EHR-Phenolyzer can tolerate some noise in the phenotype-extraction procedure, supporting its utility for genetic diseases that have clinically heterogeneous presentations.

Next, to understand the degree to which or the contexts in which the methods work, a detailed examination was performed of several illustrative cases. A case of a 15-year-old female with multiple organ-system anomalies was analyzed, including intellectual disability and skeletal dysplasia. Clinical exome sequencing identified collagen type X alpha 1 chain (COL10A1 [MIM: 120110]) as the gene with causal variants, yielding a molecular diagnosis of Schmid-type metaphyseal chondrodysplasia (MCDS [MIM: 156500]). MCDS is caused by heterozygous mutations in COL10A1 and is characterized by short stature and bowing of the long bones. For this individual, 15, 25, and 18 phenotype terms were compiled by experts, MedLEE, and MetaMap, respectively, but only five terms (spondylometaphyseal dysplasia, skeletal dysplasia, short stature, intellectual disability, and global developmental delay) were shared by all three methods. Nevertheless, this gene was ranked as #4 by Phenolyzer on all three sets of terms separately, suggesting that Phenolyzer can tolerate inaccuracies in phenotype terms and upweight highly specific terms in its scoring scheme. This example clearly demonstrates that as long as a core set of highly informative phenotype terms can be identified from EHR narratives, good ranking performance can be achieved, even if extra less-relevant terms are also included.

Another case that was analyzed involved a 13-year-old female with generalized seizures and a mutation in SCN1A. SCN1A encodes a voltage-gated sodium channel essential for the generation and propagation of action potentials and is associated with four Mendelian phenotypes in OMIM, including generalized epilepsy with febrile seizures plus type 2 (MIM: 604403), early infantile epileptic encephalopathy (MIM:607208), familial febrile seizures 3A (MIM: 604403), and familial hemiplegic migraine 3 (MIM: 609634). Surprisingly, although expert-compiled terms and MedLEE compiled terms are generally quite broad, this gene ranked as #1 and #18 on the basis of these terms, respectively. In comparison, MetaMap generated more specific phenotype terms such as “pneumonia” and “hepatic encephalopathy” (which are unrelated to SCN1A), as well as candidate disease diagnosis “autism spectrum disorders,” but SCN1A was not ranked within the top 100 genes. The above analyses highlight that EHR narratives typically contain concepts that can include both pertinent and irrelevant signs, symptoms, clinical descriptions, and clinical histories with variable levels of confidence or relevance. Thus, despite the limitations of NLP systems, the clinical information contained within the note can be extracted with the assistance of computationally enabled ontologies such as HPO and tools such as Phenolyzer. In a purely hypothetical example, the two phenotype concepts “intellectual disability” and “generalized seizure” would ideally strengthen the confidence of the computational representation of the disorder “seizure disorder” given these semantically and ontologically related concepts, improving the confidence score of finding seizure-disorder-related genes. Less-relevant concepts identified for the same individual can be regarded as peripheral to the main genetic etiology in computational phenotype-based gene prioritization. Thus, a robust relevance metric may be important for filtering out irrelevant concepts.

As discussed above, in another aspect of the implementations described herein, the combined use of exome/genome data together with phenotype data was investigated. As noted, it was shown that the combination of phenotype terms (processed by, for example, the EHR-Phenolyzer) and exome/genome data can often significantly expedite molecular diagnosis of monogenic disorders. As shown by the results from four independent cohorts, in more than half of the individuals, the genes with disease-causing mutations can be prioritized within the top 100 and in some cases even within the top ten. In clinical practice, this information can greatly reduce the effort in manually searching for candidate genes when analyzing WES data. Furthermore, as illustrated in the combined analysis of genotype and phenotype for genetic diagnosis of two individuals, the genes with causal variants were ranked as the top gene, which showcased its practical significance in clinical diagnostic settings of joint analysis of phenotype and genomic data. The validation of the approaches described herein in four independent cohorts from two different institutions also demonstrated the possibility of extending such approaches to other institutions with different informatics infrastructures.

Thus, as discussed herein with reference to FIGS. 1-7 and with respect to the various studies and investigations that took place to evaluate the efficacy of a system such as the system 100 of FIG. 1, the clinical validity of automated extraction of HPO concepts from EHR narratives for computational phenotype-driven gene prioritization was evaluated, and the results demonstrated that the proposed approach greatly facilitates the interpretation of clinical exome sequencing data. The EHR-Phenolyzer framework operates in two steps: the first uses NLP-driven phenotype-concept recognition through either the publicly available tool MetaMap or the proprietary tool MedLEE (or some other NLP tool that uses one or more ontologies of biomedical concepts), and the second utilizes the (now) open-source computational phenotype tool Phenolyzer for gene prioritization. Finally, through retrospective case studies, it was demonstrated how combined analyses of genotype and phenotype data from EHRs can expedite genetic diagnoses by using clinical exomes. It was concluded that EHR-Phenolyzer allows comprehensive utilization of deep phenotypes in EHR narratives, allows for phenotype-driven ordering and analysis of clinical exome tests, and facilitates the implementation of genomic medicine.

In previous analyses, the ranking of genes with causal variants among the ≈20,000 human genes were examined. However, in practice, clinical diagnostic labs might examine only the subset of genes known to be associated with monogenic disorders, which would make gene prioritization somewhat easier. To gain a deeper understanding of the performance of the HER Phenolyzer approach in clinical settings, the approach was assessed on its ability to rank genes among a selected list of about 5,000 OMIM genes that are known to be associated with Mendelian diseases rather than among all 20,000 genes. The results obtained showed that restricting the analysis to OMIM genes further improved the performance of EHR-Phenolyzer in detecting genes with causal variants. However, it is noted that two positive diagnoses were made on myosin heavy chain 10 (MYH10 [MIM: 160776]) and N(alpha)-acetyltransferase 15, NatA auxiliary subunit (NAA15 [MIM: 608000]), which had not yet been documented in OMIM as being associated with a Mendelian phenotype, suggesting that expanded analysis could still be warranted if OMIM-restricted analyses do not yield positive results. MYH10 and NAA15 were both discovered recently from several sequencing studies on congenital heart disease and developmental disorders.

Some features and enhancements that the various implementations described herein may include the following. The implementations can be configured to perform concept recognition procedures from structured EHR data in addition to unstructured clinical narratives, such as laboratory testing results and radiographic findings. such procedures can potentially further improve this process of automated EHR-phenotype-driven gene prioritization if these concepts are not recorded within the clinical notes. In another example, mapping from other established standard terminologies, such as Systematized Nomenclature of Medicine—Clinical Terms (SNOMED CT), to HPO may be implemented. Another example feature that can be included in some of the implementations described herein pertains to evaluating the transferability of the proposed methods to different healthcare systems that leverage different EHRs. In the current study, it was examined and confirmed that the EHR-Phenolyzer approach can be utilized in two different healthcare systems with a relatively small set of samples. This is expected to significantly expand the number of sites to be analyzed by EHR-Phenolyzer in the future and examine how to adapt the method to different settings across institutions to enable the delivery of more benefits to the broader community. Some of the implementations may also include an individual-facing Phenolyzer that allows people to enter self-reported phenotypes not captured in EHRs. With such a feature, an examination will be made as to whether individual-provided information can further improve the accuracy for gene ranking when the genomic analysts have access to such information. In order to accommodate users who speak different languages, the EHR-Phenolyzer implementations may also accommodate phenotypes entered in non-English languages. Finally, an effort to curate phenotype data in a systematic manner requires the recognition of the importance of phenotype information. As more high-quality genomic and phenotype information is collected with collaborative efforts such as the Monarch Initiative, PhenomeCentral, and HPO, it is believed that approaches driven by phenotype data will become more robust and effective. With the continuing growth of HPO, the continued development of new techniques and optimization of pre-existing NLP techniques is likely to improve term normalization across the field of genomic medicine, making these efforts easier and more effective in the future.

Unless defined otherwise, all technical and scientific terms used herein have the same meaning as commonly or conventionally understood. As used herein, the articles “a” and “an” refer to one or to more than one (i.e., to at least one) of the grammatical object of the article. By way of example, “an element” means one element or more than one element. “About” and/or “approximately” as used herein when referring to a measurable value such as an amount, a temporal duration, and the like, encompasses variations of ±20% or ±10%, ±5%, or +0.1% from the specified value, as such variations are appropriate in the context of the systems, devices, circuits, methods, and other implementations described herein. “Substantially” as used herein when referring to a measurable value such as an amount, a temporal duration, a physical attribute (such as frequency), and the like, also encompasses variations of ±20% or ±10%, ±5%, or +0.1% from the specified value, as such variations are appropriate in the context of the systems, devices, circuits, methods, and other implementations described herein.

As used herein, including in the claims, “or” as used in a list of items prefaced by “at least one of” or “one or more of” indicates a disjunctive list such that, for example, a list of “at least one of A, B, or C” means A or B or C or AB or AC or BC or ABC (i.e., A and B and C), or combinations with more than one feature (e.g., AA, AAB, ABBC, etc.). Also, as used herein, unless otherwise stated, a statement that a function or operation is “based on” an item or condition means that the function or operation is based on the stated item or condition and may be based on one or more items and/or conditions in addition to the stated item or condition.

Although particular embodiments have been disclosed herein in detail, this has been done by way of example for purposes of illustration only, and is not intended to be limiting with respect to the scope of the appended claims, which follow. Features of the disclosed embodiments can be combined, rearranged, etc., within the scope of the invention to produce more embodiments. Some other aspects, advantages, and modifications are considered to be within the scope of the claims provided below. The claims presented are representative of at least some of the embodiments and features disclosed herein. Other unclaimed embodiments and features are also contemplated.

What is claimed is:

1. A method comprising:
accessing electronic health record data for a patient;
performing natural language processing on the electronic health record data to extract biomedical concepts;
processing the biomedical concepts to obtain phenotype terms;
normalizing the phenotype terms to generate normalized phenotype terms; and
identifying based on the normalized phenotype terms one or more candidate genes responsible for one or more medical conditions causing the biomedical concepts extracted from the electronic health record data.

2. The method of claim 1, wherein processing the biomedical concepts comprises:
recognizing the biomedical concepts for disease phenotypes using semantic knowledge resources, including one or more of: UMLS (Unified Medical Language System), or HPO (Human Phenotype Ontology).

3. The method of claim 1, wherein identifying the one or more candidate genes comprises:
prioritizing the one or more candidate genes responsible for one the or more medical conditions causing the biomedical concepts extracted from the electronic health record data.

4. The method of claim 3, wherein prioritizing the one or more candidate genes comprises:
ranking the one or more identified candidate genes.

5. the method of claim 4, wherein ranking the one or more identified candidate genes comprises:
ranking the one or more candidate genes based on degree of matching between the normalized phenotype terms and respective descriptors associated with the one or more candidate genes.

6. The method of claim 1, wherein accessing the electronic health record data comprises:
determining data quality of the electronic health record data;
selecting portions of the electronic health record data based, at least in part, on the determined data quality; and
representing the selected portions of the electronic health record data in a pre-determined format for further analysis.

7. The method of claim 1, wherein processing the biomedical concepts comprises:
applying text processing to the electronic health record data at the document level and the sentence level, wherein the text processing comprises performing one or more of semantic knowledge-based or machine-learning based concept recognition to obtain the phenotype terms.

8. The method of claim 7, wherein performing the one or more of the semantic knowledge-based or machine-learning based concept recognition to obtain the phenotype terms comprises performing one or more of:
analyzing negation status associated with recognized phenotype terms,
analyzing phenotype existence for the patient or a family member of the patient to rule-out non-patient phenotype,
identifying modifiers associated with the recognized phenotype terms,
analyzing temporal properties associated with the recognized phenotype terms, or
analyzing temporal relationships among one or more phenotype terms for the patient.

9. The method of claim 1, wherein normalizing the phenotype comprises:
performing semantic knowledge-based concept normalization.

10. The method of claim 1, wherein normalizing the phenotype terms comprises:
normalizing the phenotype terms using human phenotypes ontology (HPO) definitions to generate the normalized phenotype terms.

11. The method of claim 1, further comprising:
obtaining clinical exome or genome data representative of one or more genetic profiles of the patient; and
determining at least one gene from the one or more identified candidate genes responsible for the one or more medical conditions based on the clinical exome or genome data and on the normalized phenotype terms provided to a gene-ranking tool.

12. The method of claim 1, wherein performing the natural language processing on the electronic health record data to extract biomedical concepts comprises performing the natural language processing (NLP) through multiple independent NLP platforms to produce respective multiple lists of extracted biomedical concepts;
and wherein identifying the one or more candidate genes comprises:
providing the respective multiple lists of extracted biomedical concepts to a gene-ranker to generate multiple lists of candidate genes.

13. The method of claim 12, further comprising:
ranking each of the generated multiple lists of candidate genes; and
deriving a composite ranked list of candidate genes based on the ranked multiple lists of candidate genes.

14. The method of claim 1, wherein performing natural language processing on the electronic health record data comprises:
performing natural language processing on clinical patient notes from the electronic health record data.

15. A medical analysis system comprising:
a communication module to access electronic health record data for a patient stored in a data storage device;
a natural language processing engine configured to:
perform natural language processing on the accessed electronic health record data to extract biomedical concepts;
process the biomedical concepts to obtain phenotype terms; and
normalize the phenotype terms to generate normalized phenotype terms; and
a genetic analyzer configured to identify based on the normalized phenotype terms one or more candidate genes responsible for one or more medical conditions causing the biomedical concepts extracted from the electronic health record data.

16. The system of claim 15, wherein the natural language processing engine configured to process the biomedical concepts is configured to:
recognize the biomedical concepts for disease phenotypes using semantic knowledge resources, including one or more of: UMLS (Unified Medical Language System), or HPO (Human Phenotype Ontology).

17. The system of claim 15, wherein the genetic analyzer comprises:
a gene-ranking tool to prioritize the one or more candidate genes responsible for one the or more medical conditions causing the biomedical concepts extracted from the electronic health record data.

18. The system of claim 17, wherein the gene-ranking tool configured to prioritize the one or more candidate genes is configured to:
rank the one or more identified candidate genes based on degree of matching between the normalized phenotype terms and respective descriptors associated with the one or more candidate genes.

19. The system of claim 17, wherein the genetic analyzer is further configured to:
obtain clinical exome or genome data representative of one or more genetic profiles of the patient; and
determine at least one gene from the one or more identified candidate genes responsible for the one or more medical conditions based on the clinical exome or genome data and on the normalized phenotype terms provided to the gene-ranking tool.

20. The system of claim 15, further comprising:
at least one other communication module to access the electronic health record data for the patient, and at least one other natural language processing engine configured to generate at least one other independent set of normalized phenotype terms provided to the genetic analyzer;
wherein the genetic analyzer is configured to identify the one or more candidate genes based further on the at least one other independent set of normalized phenotype terms.

21. An apparatus comprising:
means for accessing electronic health record data for a patient;
means for performing natural language processing on the electronic health record data to extract biomedical concepts;
means for processing the biomedical concepts to obtain phenotype terms;
means for normalizing the phenotype terms to generate normalized phenotype terms; and
means for identifying based on the normalized phenotype terms one or more candidate genes responsible for one or more medical conditions causing the biomedical concepts extracted from the electronic health record data.

22. Non-transitory computer readable media comprising computer instructions, executable on one or more processor-based devices, to:
access electronic health record data for a patient;
perform natural language processing on the electronic health record data to extract biomedical concepts;
process the biomedical concepts to obtain phenotype terms;
normalize the phenotype terms to generate normalized phenotype terms; and
identify based on the normalized phenotype terms one or more candidate genes responsible for one or more medical conditions causing the biomedical concepts extracted from the electronic health record data.
